(19) United States
(12) Patent Application Publication
(10) Pub. No.: US 2009/0138087 A1
(43) Pub. Date: May 28, 2009

(54) Apparatus for the Neurosurgical-Orthopedic Treatment of Human Spinal Column Pathologies

(76) Inventors: Carlo Miglietta, Milano (IT); Giuseppe Tedesco, Nardo'Le (IT)

Correspondence Address: K.F. ROSS P.C., 5683 RIVERDALE AVENUE, SUITE 203 BOX 900, BRONX, NY, 10471-0900, US

(21) Appl. No.: 11/989,440

(22) PCT Filed: Jul. 21, 2006
(86) PCT No.: PCT/IB2006/002004

§ 371 Date: Jan. 24, 2008

(30) Foreign Application Priority Data
Jul. 28, 2005 (IT) PD2005A000231

(51) Int. Cl. A61F 2/44; A61B 17/58

(52) U.S. Cl. 623/17.16; 623/17.11; 606/90

(57) ABSTRACT

Apparatus for the neurosurgical-orthopedic treatment of human spinal column pathologies, comprising an elastically yielding contrast means (6) to fit into an inter-spinal channel (c) between a pair of adjacent spinal processes (P1, P2) for the purpose of contrasting the stenosis of said channel, and constraining means (7) associated with said contrast means to be bound to said pair of adjacent spinal processes so as to hinder said contrast means from shifting outside/inside said inner-spinal channel. The peculiarity of said contrast means consists in that it comprises distancing means (8) to distance said pair of spinal processes and elastically yielding constraining means (9) to bind said distancing means, so as to achieve a graduated contrasting reaction against the stenosis of said channel.

The present invention refer to an apparatus for the neurosurgical-orthopedic treatment of human spinal column pathologies, which is particularly, yet not exclusively, useful in treating the lumbar and cervical spinal stenosis.

The lumbar and cervical stenosis is a pathology that affects the human being prevalently with advancing age. This pathology consists in a progressive reduction of the radicular inter-spinal channels existing between adjacent spinal processes of the spinal column in the lumbar, thorax and cervical region. This progressive reduction substantially reduces the space available for the passage of the blood vessels and nervous systems, leading to the following symptoms characteristic of this pathology:
muted or painful back ache diffusing from the legs;
numbness and formication of the legs, hind-legs and buttocks;
weakness, loss of balance and
falling endurance of physical activities.

These symptoms grow for instance after a walk or after the subject has remained standing for prolonged periods.

In the current medical practice, stenosis is treated in a first phase by applying non-invasive therapies such as medications, gymnastics and appropriate drugs. In a second phase it is necessary to take surgical action by implanting appropriate devices between adjacent spinal processes, which serve the purpose of enlarging and maintaining the enlarged shape of the damaged spinal channels. The subject affected by stenosis thus fully overcomes his pathological condition and recovers the normal functions associated with the spinal column.

A first type of known apparatus is made in a single piece, formed of an elastically yielding material and presents a characteristic, approximately “H” shaped form. The central horizontal element of this apparatus operates as an elastically yielding contrast means to be fitted into an inter-spinal channel between a pair of adjacent spinal processes for the purpose of contrasting the stenosis of the channel. The vertical side elements of this apparatus act as constraining means associated to the contrast means to be bound to the adjacent spinal processes so as to hinder shifting motions of the contrast means outside/inside the inter-spinal channel.

The implanting methodology of this first type of apparatus comprises a first phase of cutting the patient's rear ligament corresponding to the inter-spinal channel affected by the stenosis, and a second phase of emplacing the apparatus.

The main drawback of this apparatus consists in the fact that its implantation is particularly invasive, because in attempting to resolve the patient's pathology it is necessary to take drastic action on a healthy organ of the same. The introduction of the central horizontal element into the inter-spinal channel in fact demands the cutting of the rear ligament before introducing the same.

A second drawback of this device consists in the fact that the material employed for realizing this apparatus, generally a gel, tends to degrade in time and therefore to lose its mechanical features in time. This fact occasions numerous problems associated with the need of replacing the apparatus at regular, approximately pre-established time intervals.

A second known apparatus is essentially formed in two mutually coupled pieces made of a rigid material. In an assembled form, this apparatus defines a rigid contrast means to be fitted into an inter-spinal channel between a pair of adjacent spinal processes, so as to contrast the stenosis of the channel, and constraining means associated with the contrast means to keep the contrast means from shifting outside/inside the inter-spinal channel. In a disassembled form, this apparatus is composed of a first piece formed by the contrast means and first constraining means and by a second means formed in turn by second constraining means.

The implanting methodology of this second type of apparatus comprises a first place of emplacing the first piece of the apparatus on one side of the column, a second phase of emplacing the second piece of the apparatus on the other side of the column corresponding to the first piece, and a third phase of mutually coupling the two pieces.

The feature of this apparatus that looks more advantageous than the above type consists in the fact that its implantation is much less invasive, as no cuts of the rear ligament are needed.

The main drawback of this second type of apparatus consists in the fact that the contrast means provides no form of elastic reaction to the stresses the spinal processes are subjected to. This fact leads to abnormal stresses on the spinal processes.

The aim of the present invention is to achieve an apparatus for the neurosurgical-orthopedic treatment of human spinal column pathologies capable of resolving the mentioned drawbacks.

Within the abovementioned aim, an object of the invention consists in setting up an apparatus capable of developing a graduated contrast reaction against the stenosis of the channel receiving such apparatus.

A further object of the invention is to set up an apparatus wherein said graduated contrast reaction is mainly induced by the shape of the elastically yielding contrast means.

Another object of the invention is to set up an apparatus whose contrast reaction is enduring.

Not the last object of the invention consists in setting up an apparatus easy to use on the whole and flexible to employ, so as to ease the implanting operations.

This aim, these and other objects that will be better apparent in the following, are achieved by an apparatus for the neurosurgical-orthopedic treatment of human spinal column pathologies according to the enclosed claims.

According to first features of the invention, the elastically yielding contrast means is formed by distancing means which distance a pair of adjacent spinal processes and by elastically yielding connecting means which connect the distancing means, so as to achieve a graduated contrast reaction against the stenosis of the channel the apparatus is applied to.

According to second features of the invention, the distancing means are made of a pair of wings and the elastically yielding connecting means are constituted of an extension common to said distancing means, so that on the whole the wings and the extension form a “C” shaped body that represents a form suitable for performing said graduated elastic reaction.

According to other features of the invention, said “C” shaped body can be made of a metallic or alloy material to guarantee an enduring reaction.

According to yet further features of the invention, the apparatus is in the overall characterized by the fact of being easy to use and flexible to apply, so as to substantially ease the implantation.

Further features and advantages will be better apparent from the description of a preferred embodiment of the apparatus for the neurosurgical-orthopedic treatment of human spinal column pathologies, as shown for indicative yet not limitative purposes in the enclosed drawings, wherein:

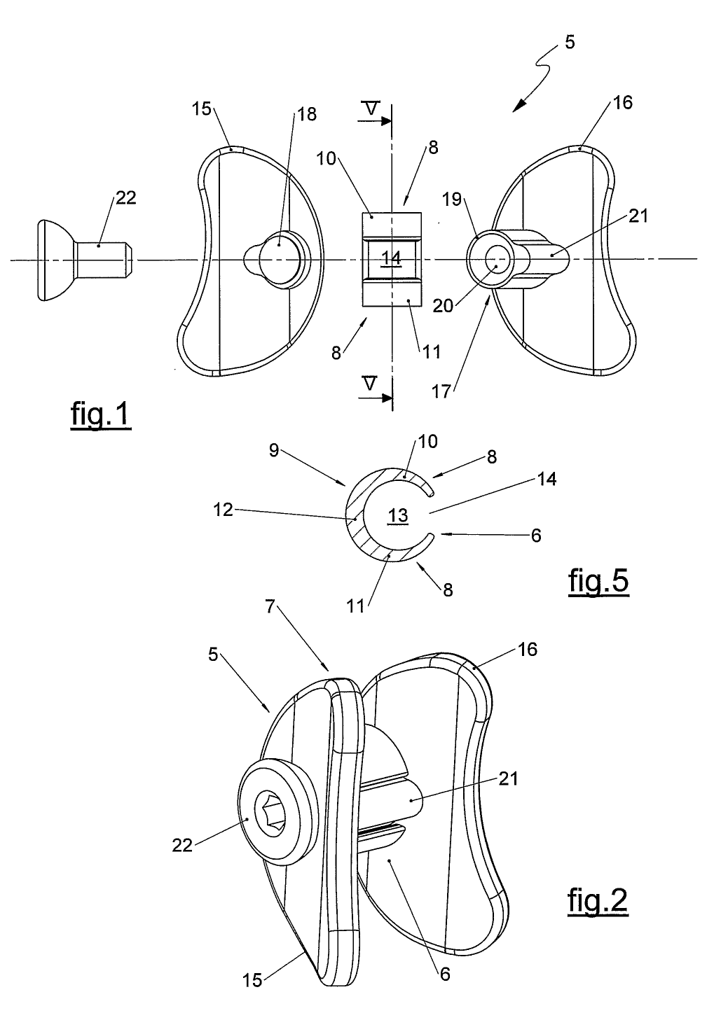

FIG. 1 represents an exploded frontal view of the apparatus according to the invention, in a disassembled condition;

FIG. 2 represents a prospective lateral/frontal view of the apparatus of the previous figure, in an assembled condition;

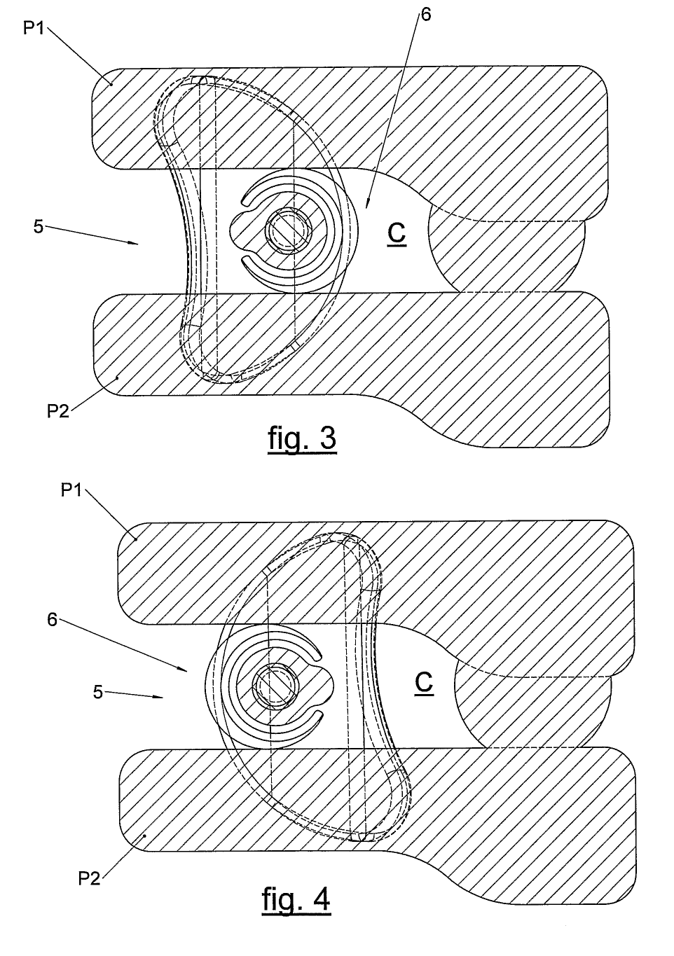

FIG. 3 represents a detailed view of a first mode of implant of the apparatus of FIGS. 1 and 2;

FIG. 4 represents a detailed view of a second mode of implant of the apparatus of FIGS. 1 and 2;

FIG. 5 represents a transversal sectional view, according to the tracing plane V-V of FIG. 1, of a part of the apparatus.

With reference to the enclosed drawings, the apparatus for the neurosurgical-orthopedic treatment of human spinal column pathologies, according to the invention, is indicated as a whole by the reference number 5. This apparatus 5 comprises an elastically yielding contrast means indicated by the reference number 6. This contrast means 6 is fitted inside an inter-spinal channel C between a pair of spinal processes P1 and P2 for the purpose of contrasting the stenosis of the channel C. The apparatus further comprises constraining means designated as a whole by the reference number 7, associated with the contrast means 6. These contrast means 7 are constrained to the spinal processes P1 and P2, so as to impede shifting motions of the contrast means 6 outside/inside the inter-spinal channel C. The contrast means is in turn constituted of distancing means, indicated as a whole by the reference number 8, which serve the purpose of distancing the pair of spinal processes P1 and P2 and by elastically yielding connecting means, indicated as a whole by the reference number 9, which have the function of connecting the mentioned distancing means 8. In this manner, the distancing means 8 and the connecting means 9 achieve a contrast reaction graduated to the stenosis of the channel C. These distancing means 8 are appropriately disposed facing each other and placed in contact with the spinal processes P1 and P2. In detail, said distancing means 8 are constituted of a pair of wings designated by the reference numbers 10 and 11. These wings 10 and 11 are advantageously formed in a curved manner and placed in contact with the spinal processes P1 and P2 in the regions of maximum curvature. The connecting means 9 are in turn constituted of an extension 12 common to both distancing means 8. The peculiar characteristic of this extension consists in a thickness growing in a direction toward their central portion. This characteristic determines a variation of the elastic reaction depending on the opposite pressure and traction stresses applied to the distancing means 8. In the overall, therefore, the wings 10 and 11 together with the extension 12 essentially define a “C” type body which, in turn, defines a central cylindrical space 13 and an opening 14 of the space 13 communicating with the exterior. This “C” type body is advantageously realized by employing for instance a metallic alloy belonging to the family of titanium alloys that present fair biocompatibility characteristics, fair elastic properties and high degradation resistance. The constraining means 7 are in turn constituted of a pair of platelets 15 and 16 placed facing each other, and of a connecting element 17 that connects the mentioned platelets 15 and 16. The latter are profiled approximately to the shape of a laid-down “V” and fitted with their opposite extremities bent in a divergent manner. This particular configuration of the platelets 15 and 16 reproduces the lateral profile of the spinal processes P1 and P2 so as to adapt themselves to them once the apparatus 5 has been implanted. Moreover, the platelet designated by the reference number 15 is fitted with a hole 18 provided next to the apex of the same platelet 15. The connecting element 17 is in turn formed by a cylindrical appendix 19 that extends in an orthogonal direction from the platelet 16, devoid of a hole, to the zone next to its apex toward the other platelet, for a length not inferior to that of the contrast means 6. Said connecting means 17 is also fitted with a central hole 20 applied in an axial direction on the appendix 19 and internally threaded, and provided with a bump 21 that extends in a frontal direction from the mantle of the mentioned appendix 19. This appendix 19 is also received inside a central cylindrical space 13 and the bump 21 projects to the outside across the mentioned opening 14. In conclusion, said connecting element 17 comprises a locking key 22 inserted into the hole 18 provided in the platelet 15 and threaded into the central hole 20 provided on the appendix 19.

With particular reference to the FIGS. 3 and 4, the procedure of implanting the apparatus 5 occurs as follows. The first stage consists in preparing the patient for the implantation. The second phase consists in selecting the contrast means 6 among a series of contrast means 9 of different size, depending on the size of the patient's inter-spinal channel C. The third phase consists in introducing the contrast means 6 inside the inter-spinal channel 6 while disposing it transversally to the inter-spinal channel C and according to one of the configuration s shown in the FIGS. 3 and 4. The fourth phase consists in the complete lateral introduction of the connecting element 17 into the central cylindrical space 13. The fifth phase consists in the final assembly of the apparatus 5, wherein the platelet 15 is coupled to the connecting element 17, the introduction of the locking screw 22 into the central hole 18 of the platelet 15, and the complete tightening down of the screw 22 into the central hole 20.

It has in practice been found that the apparatus for the treatment of neurosurgical-orthopedic pathologies of the human spinal column achieves the pre-established aim and objects. In particular, the apparatus has proved to be capable of performing a graduated contrast reaction against the stenosis of the channel the apparatus is applied to. Moreover, it has been found that graduated contrast reaction performed by the elastically yielding contrast means is above all due to its form. It has further been found that the graduated contrast reaction performed by the elastically yielding contrast means is durable. Finally, the apparatus has on the whole been found easy to use and flexible to employ, so as to ease the implanting operations.

The structure according to the invention is susceptible to numerous modifications and variants, all of which fall within the scope of the same inventive concept.

In a practical embodiment, the materials employed, the forms, dimensions and executive details may differ from those indicated above, but be technically equivalent to them, without thereby abandoning the scope of the invention.

1. Apparatus for the neurosurgical-orthopedic treatment of human spinal column pathologies, comprising an elastically yielding contrast means to fit into an inter-spinal channel between a pair of adjacent spinal processes for the purpose of contrasting the stenosis of said channel, and constraining means associated with said contrast means to be constrained to said pair of adjacent spinal processes so as to hinder said contrast means from shifting outside/inside said inter-spinal channel wherein said contrast means comprises distancing means to distance said pair of spinal processes and elastically yielding connecting means to connect said distancing means so as to achieve a graduated contrasting reaction against the stenosis of said channel.

2. Apparatus, according to claim 1 wherein said distancing means are arranged to mutually face each other and be placed in contact with said spinal processes.

3. Apparatus, according to claim 1 wherein said distancing means are constituted of a pair of wings.

4. Apparatus, according to claim 3 wherein said wings are shaped in a curved manner.

5. Apparatus, according to claim 3 wherein said wings are placed in contact with said spinal processes in the regions of maximum curvature.

6. Apparatus, according to claim 1 wherein said elastically yielding connecting means are constituted of an extension common to both said distancing means.

7. Apparatus, according to claim 1 wherein said extension has a growing thickness in the direction toward the central region.

8. Apparatus, according to claim 3 wherein said pair of wings and said extension essentially define a “C” shaped body that in turn defines a central cylindrical space and an opening to communicate with the outside.

9. Apparatus, according to claim 1 wherein said “C” shaped body is realized of a material whose physical-chemical characteristics are durable and constant in time.

10. Apparatus, according to claim 1 wherein according to a first modality of the implant, said “C” shaped body is arranged with said communication opening turned to the opposite side of said spinal column and that, according to a second modality of the implant, said “C” shaped body is arranged with said communicating opening turned toward said spinal column.

11. Apparatus, according to claim 1 wherein said constraining means comprise a pair of platelets arranged to face each other and a connecting element to connect said platelets.

12. Apparatus, according to claim 11 wherein said platelets are shaped approximately like a laid-down “V” and are provided with opposed terminal regions bent in a divergent fashion.

13. Apparatus, according to claim 11 wherein one platelet of said pair of platelets comprises a hole provided next to the apex of the same platelet.

14. Apparatus, according to claim 11 wherein said connecting element comprises a cylindrical appendix extending in an orthogonal direction from the region next to the apex of said platelet devoid of said hole toward the other platelet and has a length not inferior to that of said contrast means.

15. Apparatus, according to claim 14 wherein said connecting element is fitted with a central hole provided in an axial direction on said appendix and internally threaded, and provided with a bump that extends frontally from the mantle of said appendix.

16. Apparatus, according to claim 14 wherein said appendix is received inside said space obtained in said contrast means and said bump projects toward the outside across said opening.

17. Apparatus, according to claim 11 wherein said connecting element comprises a locking screw inserted in said hole on said platelet and is engaged in said central hole by threading it into said central hole in said appendix.
